(19) United States
(12) Patent Application Publication
(10) Pub. No.: US 2023/0367394 A1
(43) Pub. Date: Nov. 16, 2023

(54) DATA TRANSFER BETWEEN USER-WORN DEVICES

(71) Applicant: Arm Limited, Cambridge (GB)

(72) Inventors: Emre OZER, Cambridge (GB); Parameshwarappa Anand Kumar SAVANTH, Cambridge (GB); Jedrzej KUFEL, Cambridge (GB); Sahan Sajeewa Hiniduma Udugama GAMAGE, Cambridge (GB); John Philip BIGGS, Cambridge (GB)

(21) Appl. No.: 18/313,102

(22) Filed: May 5, 2023

(30) Foreign Application Priority Data
May 12, 2022 (GB) 2206956.1

(51) Int. Cl. G06F 3/01; A61B 5/256; A61B 5/00

(52) U.S. Cl. CPC G06F 3/015; A61B 5/256; A61B 5/7285

(57) ABSTRACT

Wearable devices, systems of wearable devices, and methods of operating the same are disclosed. A first wearable device worn in contact with the user’s skin monitors the user and comprises a transmission electrode in contact with the user’s skin. A second wearable device comprises a reception electrode worn in contact with the user’s skin. The first wearable device can apply an alert signal to the transmission electrode and measures a transmission current at the transmission electrode. The second wearable device monitors an electrical status of the reception electrode and when the alert signal is detected applies an alert response signal to the receiver electrode. The first wearable device identifies application of the alert response signal to the receiver electrode by measurement of a variation of the transmission current at the transmission electrode whilst the alert signal is applied to the transmission electrode.

### CROSS-REFERENCE TO RELATED APPLICATIONS

This application claims priority pursuant to 35 U.S.C. 119(a) to British Application No. 2206956.1, filed May 12, 2022, which application is incorporated herein by reference in its entirety.

### FIELD

The present techniques relate to wearable devices. In particular, it relates to communication and data transfer between user-worn devices.

### BACKGROUND

User wearable devices are now widespread and may take a great variety of forms. Some wearable devices, such as a smartwatch, are provided with considerable data processing and storage capability, as well as the ability to communicate with other devices, e.g. to be coupled to a user’s smartphone by WiFi and Bluetooth. However, some user wearable devices may also have notably less data processing, storage, and communication. This may be for a number of reasons such as cost and disposability. For example, in a medical context a simple patch, such as an ECG patch, may be provided with some limited ability to perform data processing, but in order to keep the cost of the single use ECG patch to an acceptable level, the ECG patch may rely on a cabled connection for the transfer of any data which it gathers or generates to a further device.

### SUMMARY

At least some examples provide a system of wearable devices to be worn by a user comprising:
a first wearable device configured to be worn in contact with the user’s skin and to measure at least one physiological quantity with respect to the user, wherein the first wearable device comprises a transmission electrode configured to be worn in contact with the user’s skin; and
a second wearable device configured to be worn by the user, wherein the second wearable device comprises a reception electrode configured to be worn in contact with the user’s skin,
wherein the first wearable device is configured to respond to a trigger event determined by the first wearable device with reference to the at least one physiological quantity by:
   applying an alert signal to the transmission electrode; and
   measuring a transmission current at the transmission electrode,
   wherein the second wearable device is configured to monitor an electrical status of the reception electrode and in response to a determination that the electrical status is indicative of the alert signal being transmitted by the first wearable device to:
      apply an alert response signal to the receiver electrode, wherein the alert response signal is derived from the alert signal received at the reception electrode,
      and wherein the first wearable device is configured to determine whether the alert response signal has been applied to the receiver electrode by the second wearable device in dependence on a measurement of a variation of the transmission current at the transmission electrode whilst the alert signal is applied to the transmission electrode.

At least some examples provide a wearable device configured to be worn in contact with a user’s skin comprising:
measurement circuitry configured to measure at least one physiological quantity with respect to the user;
processing circuitry configured to perform data processing on physiological signals received from the measurement circuitry; and
a transmission electrode configured to be worn in contact with the user’s skin,
wherein the processing circuitry is configured to detect to a trigger event in dependence on the physiological signals received from the measurement circuitry and in response to cause:
   an alert signal to be applied to the transmission electrode; and
   measurement of a transmission current at the transmission electrode,
   and wherein the wearable device is configured to determine whether the alert signal has been received by a further wearable device worn by the user in dependence on a measurement of a variation of the transmission current at the transmission electrode whilst the alert signal is applied to the transmission electrode.

At least some examples provide a wearable device configured to be worn by a user comprising:
a reception electrode configured to be worn in contact with the user’s skin;
voltage monitoring circuitry to monitor an electrical status of the reception electrode; and
voltage application circuitry to apply a response voltage to the reception electrode,
wherein in response to a determination that the electrical status is indicative of an alert signal being transmitted by a further wearable device worn by the user to:
   apply an alert response signal to the receiver electrode, wherein the alert response signal is derived from the alert signal received at the reception electrode and is generated to cause a variation in a transmission current for application of the alert signal at a transmission electrode of the further wearable device.

At least some examples provide a method of operating a system of wearable devices worn by a user comprising:
wearing a first wearable device in contact with the user’s skin, wherein the first wearable device comprises a transmission electrode configured to be worn in contact with the user’s skin;
measuring with the first wearable device at least one physiological quantity with respect to the user;
wearing a second wearable device, wherein the second wearable device comprises a reception electrode configured to be worn in contact with the user’s skin;
responding to a trigger event determined by the first wearable device with reference to the at least one physiological quantity by:
   applying an alert signal to the transmission electrode; and
   measuring a transmission current at the transmission electrode;
   monitoring an electrical status of the reception electrode and in response to a determination that the electrical status is indicative of the alert signal being transmitted by the first wearable device:
      applying an alert response signal to the receiver electrode, wherein the alert response signal is derived from the alert signal received at the reception electrode; and
      determining whether the alert response signal has been applied to the receiver electrode by the second wearable device in dependence on a measurement of a variation of the transmission current at the transmission electrode whilst the alert signal is applied to the transmission electrode.

### BRIEF DESCRIPTION

The present techniques will be described further, by way of example only, with reference to embodiments thereof as illustrated in the accompanying drawings, to be read in conjunction with the following description, in which:

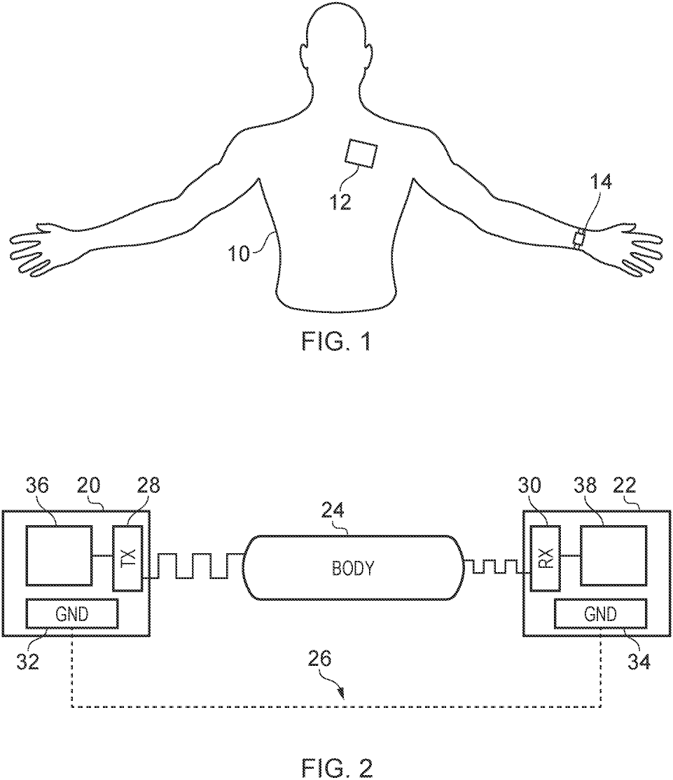

FIG. 1 schematically illustrates a user wearing a first wearable device in the form of a skin-adhesive patch and a second wearable device in the form of a smartwatch in accordance with some examples;

FIG. 2 schematically illustrates communication between a first wearable device and a second wearable device through the medium of the user’s body in accordance with some examples;

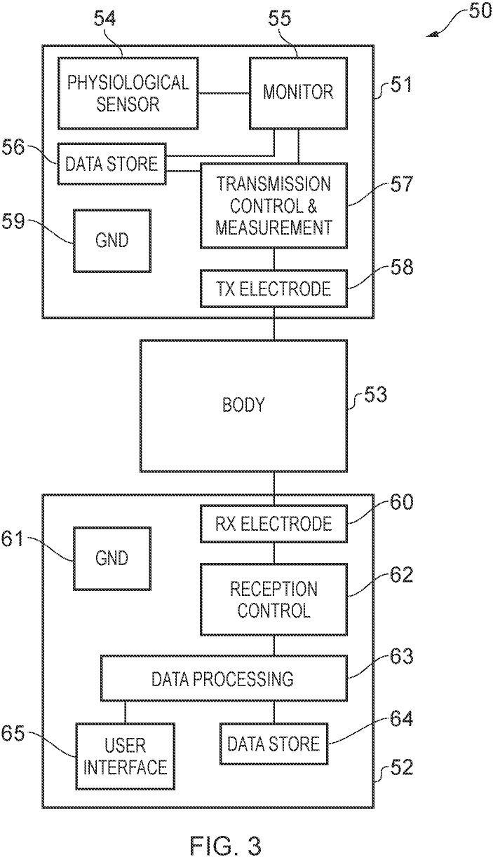

FIG. 3 schematically illustrates a system of wearable devices comprising a first wearable device and a second wearable device configured to communicate via the medium of the user’s body in accordance with some examples;

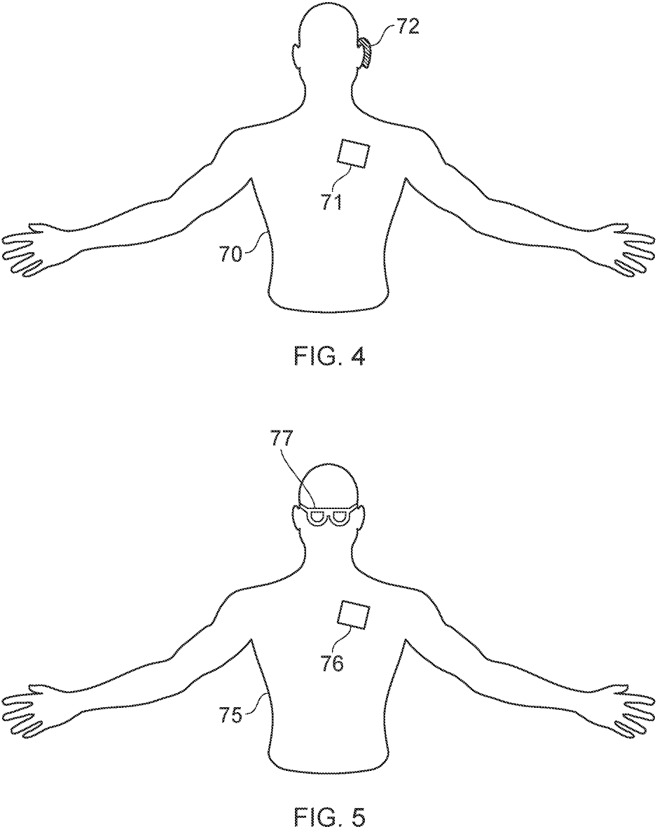

FIG. 4 schematically illustrates a user wearing a first wearable device in the form of a skin adhesive patch and a second wearable device in the form of an ear-mounted device in accordance with some examples;

FIG. 5 schematically illustrates a user wearing a first wearable device in the form of the skin adhesive patch and a second wearable device in the form of a pair of glasses in accordance with some examples;

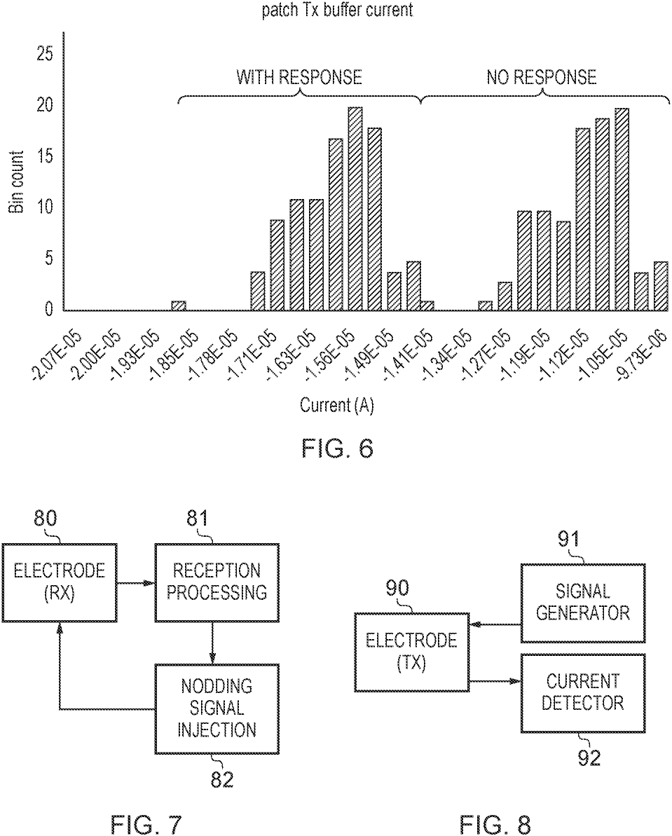

FIG. 6 illustrates simulation of an example distribution of buffer currents at the transmission buffer of a user-worn patch when a further user worn device is responding or not responding in accordance with the present techniques in some examples;

FIG. 7 schematically illustrates some components associated with a reception electrode in accordance with some examples;

FIG. 8 schematically illustrates some components associated with a transmission electrode in accordance with some examples;

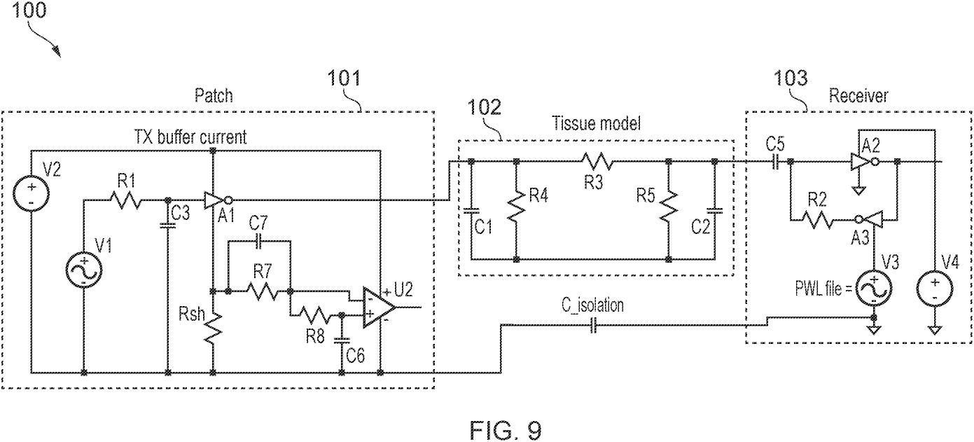

FIG. 9 schematically illustrates in more detail the configuration of a first wearable device in the form of a skin patch, a tissue model representing the user’s body, and a second wearable receiver device in accordance with some examples;

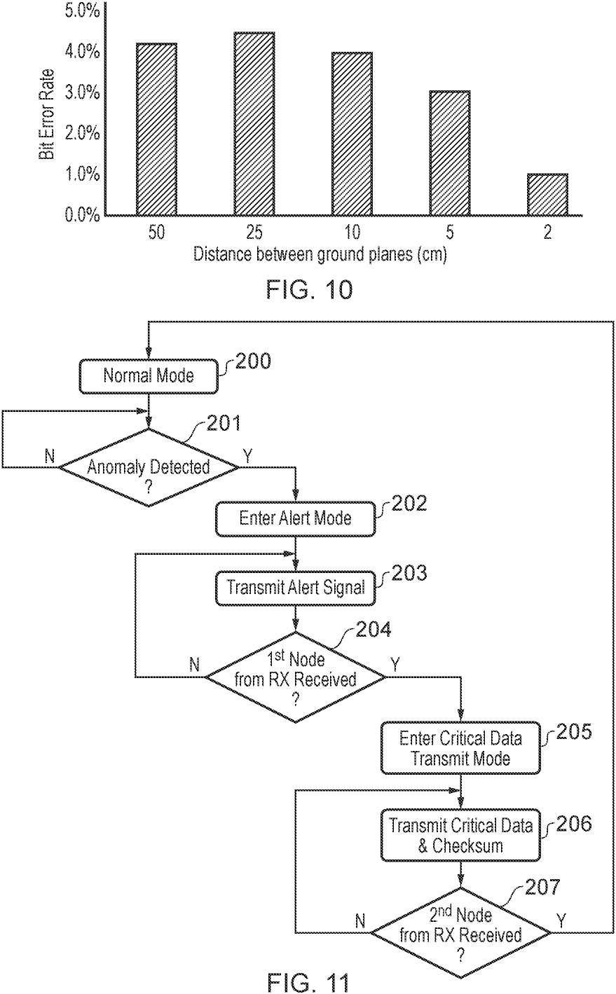

FIG. 10 illustrates an example distribution of bit error rates measured in association with body-mediated communication between a first user wearable device and a second user wearable device for a range of distances between the two devices in accordance with some examples;

FIG. 11 is a flow diagram showing a sequence of steps which are taken in accordance with the method of some examples; and

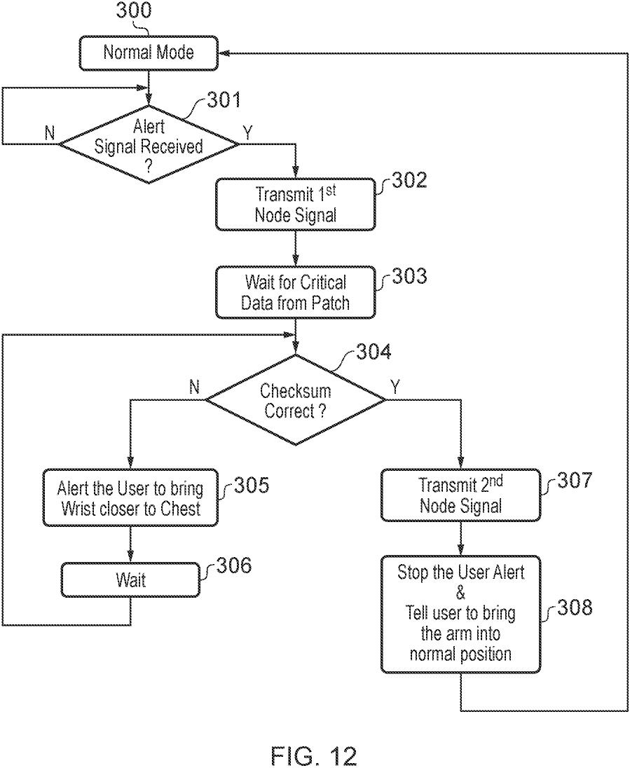

FIG. 12 is a flow diagram showing a sequence of steps which are taken in accordance with the method of some examples.

### DETAILED DESCRIPTION

In one example herein there is a system of wearable devices to be worn by a user comprising:
a first wearable device configured to be worn in contact with the user’s skin and to measure at least one physiological quantity with respect to the user, wherein the first wearable device comprises a transmission electrode configured to be worn in contact with the user’s skin; and
a second wearable device configured to be worn by the user, wherein the second wearable device comprises a reception electrode configured to be worn in contact with the user’s skin,
wherein the first wearable device is configured to respond to a trigger event determined by the first wearable device with reference to the at least one physiological quantity by:
   applying an alert signal to the transmission electrode; and
   measuring a transmission current at the transmission electrode,
   wherein the second wearable device is configured to monitor an electrical status of the reception electrode and in response to a determination that the electrical status is indicative of the alert signal being transmitted by the first wearable device to:
   apply an alert response signal to the receiver electrode, wherein the alert response signal is derived from the alert signal received at the reception electrode,
   and wherein the first wearable device is configured to determine whether the alert response signal has been applied to the receiver electrode by the second wearable device in dependence on a measurement of a variation of the transmission current at the transmission electrode whilst the alert signal is applied to the transmission electrode.

The present techniques are based on the realisation that there are a range of situations in which it may be desirable for the first wearable device worn by a user to be a low-cost, low-complexity device, to the extent that it becomes economically viable for the device even to be a single use, disposable item, and yet nevertheless for the first wearable device to be able reliably to communicate data to a further user-worn device. Hence, one object of the present techniques is to provide a system of wearable devices comprising a first wearable device worn in contact with the user’s skin and a second wearable device configured to be worn by the user, wherein the first wearable device can measure at least one physiological quantity with respect to the user and can reliably communicate data associated with that measurement to the second wearable device. A particular focus of the present techniques is to enable the first wearable device to be able to determine when the second wearable device has received a transmission sent from the first wearable device, without having to provide the supporting components for fully symmetric communication (i.e. equivalent transmission and reception capability for both the first wearable device and the second wearable device). This is achieved in accordance with the present techniques based on the recognition that, when the first wearable device is applying a given signal to a transmission electrode worn in contact with the user’s skin, the second wearable device can affect the transmission current at the transmission electrode in a manner which can be detected by the first wearable device. In particular, by means of the effect it has on the transmission current at the transmission electrode it can be determined by the first wearable device whether the second wearable device is receiving and responding to the received signal at the second wearable device’s receiver electrode, and this can be used as a communication signal for the second wearable device to acknowledge receipt of the given signal. Accordingly, a system is provided in which on the one hand, when the first wearable device is applying an alert signal to its transmission electrode, it also measures a transmission current at the transmission electrode. On the other hand, the second wearable device monitors an electrical status of the reception electrode (e.g. its voltage or the current flow through it) and thus can determine when the alert signal is being transmitted by the first wearable device. In response, the second wearable device then applies an alert response signal to the receiver electrode, where the alert response signal is derived from the alert signal received at the reception electrode. The alert response signal may be derived from the alert signal in a variety of ways. That is, it has been found that there are a number of ways in which the second wearable device can cause the transmission current at the transmission electrode of the first wearable device to vary in such a manner as to be reliably identifiable as an acknowledgement of receipt by the second wearable device of the alert signal from the first wearable device. For example, the second wearable device could directly mimic the alert signal, thus lowering the transmission current at the transmission electrode since the first wearable device has to work less to “push” the alert signal from the transmission electrode through the user’s skin to the reception electrode. Conversely, the second wearable device could directly invert the alert signal, thus directly countering the work that the first wearable device is doing to “push” the alert signal from the transmission electrode through the user’s skin to the reception electrode and hence increasing the transmission current at the transmission electrode. Other variants are possible. This provides the system with a reliable mechanism for the second wearable device to acknowledge that it has received the alert signal sent from the first wearable device, without having to provide explicit signal reception capability at the first wearable device, a simple, low-cost, low complexity device.

Generally speaking, the above described techniques provide a useful mechanism for signal acknowledgement from second wearable device to a first wearable device, of particular applicability where there is a desire to keep the first wearable device of limited constructional complexity. A further factor which may be of relevance in the context of two user worn devices which communicate via skin contact electrodes, is that the data transmission reliability of that communication medium can be somewhat fallible. Accordingly, data transmission from the first wearable device to the second wearable device may be arranged to take place in a staged manner, whereby communication between the two is first established by the first wearable device sending the alert signal to the second wearable device and the second wearable device acknowledging receipt of the alert signal by means of the application of the alert response signal to the receiver electrode. Thereafter, the first wearable device may attempt to transmit a set of data to the second wearable device.

Thus, in accordance with some examples the first wearable device is responsive to the determination that the alert response signal has been applied to the receiver electrode by the second wearable device to:
cease applying the alert signal to the transmission electrode; and
initiate transmission of user data derived from measurement of the at least one physiological quantity, wherein the transmission of the user data is encoded in a user data transmission signal applied to the transmission electrode,
wherein the user data transmission signal further encodes checksum data for the user data.

Accordingly, having established that the communication path to the second wearable device is currently viable (at least to the extent that the second wearable device was able to receive and acknowledge the alert signal), the first wearable device then attempts to transmit the user data. The provision of the checksum data with the user data enables the second wearable device to determine if it has correctly received the user data.

Hence in some examples the second wearable device is configured to further monitor the electrical status of the reception electrode and to receive the user data transmission signal,

wherein the second wearable device is configured to data process the user data and the checksum data derived from the user data transmission signal and thereby to generate a determination of whether the user data transmission signal has been received correctly.

In some examples the second wearable device is configured to communicate the determination to the user.

The inventors of the present techniques have found that there can be a noticeable difference in the error rate of communications between two such user worn devices communicating via skin contact electrodes depending on the distance which the two user worn devices are from one another. In order for the first wearable device to reliably communicate data to a second wearable device, it is therefore preferable for the two devices to be held in relatively close proximity to one another. However, the distance between the two devices is under the control of the user wearing them, who generally will not be aware of when one device wishes to communicate with the other. In this context the inventors of the present techniques have identified that the above described mechanism for the second wearable device to acknowledge receipt of an alert signal sent by the first wearable device can be usefully used to enable the first wearable device only in the first instance to transmit the alert signal to the second wearable device, until it has received the receipt acknowledgement from the second wearable device. This then provides the opportunity for the second wearable device, in addition to acknowledging receipt to the first wearable device, to also signal to the user that the first wearable device needs to transmit some data to the second wearable device. In response the user can, for example, then bring the second wearable device into closer proximity with the first wearable device in order to improve the data transmission fidelity. Once the second wearable device determines that it has received the user data correctly based on the checksum data, it can then communicate this fact to the user, indicating that the use can now move the two wearable devices away from one another. To take just one example, where the first wearable device is a skin patch, say worn on the chest, and the second wearable device is a smartwatch, the smartwatch can firstly determine that the skin patch has alerted it that it has user data to transmit, and the smartwatch can alert the user to bring the smartwatch into closer proximity with the skin patch, and then once the user data has been successfully transferred from the skin patch to the smartwatch, the smartwatch can signal the successful data transmission to the user, who can then move the smartwatch away from the skin patch.

In some examples the second wearable device is responsive to the determination indicating that the user data transmission signal has been received correctly to:
apply a user data response signal to the receiver electrode, wherein the user data response is derived from the user data transmission signal received at the reception electrode,
and wherein the first wearable device is configured to determine whether the user data response signal has been applied to the receiver electrode by the second wearable device in dependence on a measurement of a variation of the transmission current at the transmission electrode whilst the user data transmission signal is applied to the transmission electrode.

In some examples the first wearable device is responsive to the determination that the user data response signal has been applied to the receiver electrode by the second wearable device to:

cease applying the user data response signal to the transmission electrode.

A dynamic process of alerting the user to the need bring the second wearable device and the first wearable device into closer proximity can thus be supported, whereby in some examples the second wearable device is responsive to the determination indicating that the user data transmission signal has not been received correctly to:

generate a user advisory signal to indicate that the user should bring the second wearable device and the first wearable device into closer proximity.

Accordingly, in some examples the second wearable device is responsive to a subsequent determination indicating that the user data transmission signal has been received correctly after having generated the user advisory signal to:
cease generation of the user advisory signal; and/or
generate a user confirmation signal to indicate that the user data transmission signal has been correctly received so that the user need no longer hold the second wearable device and the first wearable device in close proximity.

As mentioned above, the second wearable device may be arranged to generate the alert response signal in a variety of ways in dependence on the alert signal received at the reception electrode. That is the alert response signal may be derived in a variety of ways from the alert signal. In some examples the second wearable device is configured to generate the alert response signal to phase match and amplitude match the alert signal received at the reception electrode. Conversely in some examples the second wearable device is configured to generate the alert response signal to amplitude match and phase shift the alert signal received at the reception electrode. In some examples the second wearable device is configured to generate the alert response signal with an opposite phase to the alert signal received at the reception electrode.

The first wearable device may take a wide variety of forms, but it will be appreciated that the present techniques are particularly applicable to configurations in which it is desirable for the first wearable device to be of simple, non-complex construction. Thus in some examples the first wearable device is configured as a single-use device for temporarily wearing by the user for the purpose of monitoring the at least one physiological quantity with respect to the user.

The physical construction of the first wearable device may take a wide variety of forms, but in some examples the first wearable device comprises at least one of:
electronic components arranged on a flexible substrate;
printed electronic components; and
an energy harvesting component to provide a source of electrical power to the first wearable device.

The second wearable device may also take a wide variety of forms, but in some examples the second wearable device is one of:
a smartwatch;
an earpiece; and
an ocular device.

In one example herein there is a wearable device configured to be worn in contact with a user’s skin comprising:
measurement circuitry configured to measure at least one physiological quantity with respect to the user;
processing circuitry configured to perform data processing on physiological signals received from the measurement circuitry; and
a transmission electrode configured to be worn in contact with the user’s skin,
wherein the processing circuitry is configured to detect to a trigger event in dependence on the physiological signals received from the measurement circuitry and in response to cause:
   an alert signal to be applied to the transmission electrode; and
   measurement of a transmission current at the transmission electrode,
   and wherein the wearable device is configured to determine whether the alert signal has been received by a further wearable device worn by the user in dependence on a measurement of a variation of the transmission current at the transmission electrode whilst the alert signal is applied to the transmission electrode.

In one example herein there is a wearable device configured to be worn by a user comprising:
a reception electrode configured to be worn in contact with the user’s skin;
voltage monitoring circuitry to monitor an electrical status of the reception electrode; and
voltage application circuitry to apply a response voltage to the reception electrode,
wherein in response to a determination that the electrical status is indicative of an alert signal being transmitted by a further wearable device worn by the user to:
   apply an alert response signal to the receiver electrode, wherein the alert response signal is derived from the alert signal received at the reception electrode and is generated to cause a variation in a transmission current for application of the alert signal at a transmission electrode of the further wearable device.

In one example herein there is a method of operating a system of wearable devices worn by a user comprising:
wearing a first wearable device in contact with the user’s skin, wherein the first wearable device comprises a transmission electrode configured to be worn in contact with the user’s skin;
measuring with the first wearable device at least one physiological quantity with respect to the user;
wearing a second wearable device, wherein the second wearable device comprises a reception electrode configured to be worn in contact with the user’s skin;
responding to a trigger event determined by the first wearable device with reference to the at least one physiological quantity by:
applying an alert signal to the transmission electrode; and
measuring a transmission current at the transmission electrode;
monitoring an electrical status of the reception electrode and in response to a determination that the electrical status is indicative of the alert signal being transmitted by the first wearable device:
   applying an alert response signal to the receiver electrode, wherein the alert response signal is derived from the alert signal received at the reception electrode; and
   determining whether the alert response signal has been applied to the receiver electrode by the second wearable device in dependence on a measurement of a variation of the transmission current at the transmission electrode whilst the alert signal is applied to the transmission electrode.

In some examples the method further comprises:
in response to the determination that the alert response signal has been applied to the receiver electrode:
   ceasing applying the alert signal to the transmission electrode; and
   initiating transmission from the first wearable device of user data derived from measurement of the at least one physiological quantity, wherein the transmission of the user data is encoded in a user data transmission signal applied to the transmission electrode,
   wherein the user data transmission signal further encodes checksum data for the user data.

In some examples the method further comprises:
further monitoring the electrical status of the reception electrode and receiving the user data transmission signal;
data processing the user data and the checksum data derived from the user data transmission signal; and
generating a determination of whether the user data transmission signal has been received correctly.

In some examples the method further comprises:

communicating the determination to the user.

In some examples the method further comprises:
in response to the determination indicating that the user data transmission signal has been received correctly:
   applying a user data response signal to the receiver electrode, wherein the user data response is derived from the user data transmission signal received at the reception electrode; and
   determining at the first wearable device whether the user data response signal has been applied to the receiver electrode by the second wearable device in dependence on a measurement of a variation of the transmission current at the transmission electrode whilst the user data transmission signal is applied to the transmission electrode.

In some examples the method further comprises:

in response to the determination at the first wearable device that the user data response signal has been applied to the receiver electrode by the second wearable device, ceasing applying the user data response signal to the transmission electrode.

In some examples the method further comprises:

in response to the determination at the second wearable device indicating that the user data transmission signal has not been received correctly, generating at the second wearable device a user advisory signal to indicate that the user should bring the second wearable device and the first wearable device into closer proximity.

In some examples the method further comprises:
in response to a subsequent determination at the second wearable device indicating that the user data transmission signal has been received correctly after having generated the user advisory signal:
   ceasing generation of the user advisory signal; and/or
   generating a user confirmation signal to indicate that the user data transmission signal has been correctly received so that the user need no longer hold the second wearable device and the first wearable device in close proximity.

Some particular embodiments are now described with reference to the figures.

FIG. 1 schematically illustrates a user 10 wearing a first wearable device in the form of a skin-adhesive patch 12 and a second wearable device in the form of a smartwatch 14 in accordance with some examples. The skin-adhesive patch 12 may take a variety of forms, but in the example shown in FIG. 1 is configured as a single use electrocardiogram (ECG) patch, positioned in order to be able to detect electrical activity of the user’s heart. Although it is a single use skin patch, the patch 12 is nonetheless provided with a limited data processing capacity, whereby a number of electronic components are arranged on a flexible substrate to provide a basic data processor, capable of taking signals detected from the user’s heart, and identifying certain predefined sequences of signals, which it has been determined would be of benefit to the user to be brought to the attention of the user’s cardiologist (e.g. arrhythmia). The patch may for example sense the signals from the heart, filter out noise, digitise the signal, and possibly other minor data processing with respect to the signal. One or more of the components may be printed electronic components. The patch may be provided with a minimally sized battery to power this simple system, or in some examples may comprise an energy harvesting component to provide a source of electrical power. The patch has a transmission electrode in contact with the user’s skin. The smartwatch 14 has a reception electrode configured to be worn in contact with the user’s skin, for example provided on the reverse face of the watch.

FIG. 2 schematically illustrates communication between a first wearable device and a second wearable device through the medium of the user’s body in accordance with some examples. The skin-adhesive patch 12 and smartwatch 14 of FIG. 1 are examples of these devices. The first wearable device 20 and the second wearable device 22 are configured to communicate through that conductive medium which the user’s own body 24 provides. The general principle of the present techniques is based on the conductive forward path that the user’s body 24 provides between the first wearable device 20 and the second wearable device 22, where a parasitic return path 26 completes the circuit. The first wearable device 20 has a transmission electrode 28 which is in contact with the user’s skin and similarly the second wearable device 22 has a reception electrode 30 which is also in contact with the user’s skin. A floating ground nodes 32 and 34 respectively of the first wearable device 20 and the second wearable device 22 couple to the parasitic return path 26. Each of the first wearable device 20 and the second wearable device 22 is further provided with data processing circuitry 36 and 38 respectively, which control the communication via the transmission electrode 28 and the reception electrode 30. This communication is described in more detail with reference to the figures which follow. However, in general outline, the first wearable device 20 is configured to respond to a trigger event which it determines with reference to at least one physiological quantity which it measures. In the example of the skin-adhesive ECG patch 12 of FIG. 1, this might be a particular pattern of electrical activity of the user’s heart indicative of atrial fibrillation. In response, the data processing circuitry 36 of the first wearable device 20 causes a predetermined alert signal to be applied to the transmission electrode 28. Once this alert signal is being applied to the transmission electrode 28, the data processing circuitry 36 monitors the transmission current. The second wearable device is configured to continuously monitor the electrical status of the reception electrode and in particular to monitor for an electrical status (voltage and/or current) indicative of the alert signal being transmitted by the first wearable device. For example, this could be an oscillating electrical signal with a predetermined frequency. When this is detected, the data processing circuitry 38 of the second wearable device 22 causes a predetermined alert response signal to be applied to the receiver electrode. The alert response signal is derived from the alert signal received at the reception electrode, with the explicit purpose of affecting the transmission current which the data processing circuitry 36 is monitoring at the transmission electrode 28. The first wearable device 20 determines whether the alert response signal has been applied to the receiver electrode 30 by the second wearable device 22 in dependence on a variation of the transmission current measured at the transmission electrode 28 whilst the alert signal is being applied to the transmission electrode 28. This mechanism of acknowledgment by second wearable device 22 of the alert signal sent from the first wearable device 20 is also referred to herein as “nodding”.

FIG. 3 schematically illustrates a system 50 of wearable devices comprising a first wearable device 51 and a second wearable device 52 configured to communicate via the medium of the user’s body 53 in accordance with some examples. The first wearable device 51 comprises a physiological sensor 54 and monitoring circuitry 55 arranged to monitor signals generated by the physiological sensor 54. Corresponding physiological data generated by the monitor circuitry 55 can be stored in the data store 56 and all passed directly to the transmission control and measurement circuitry 57. Accordingly, the first wearable device 51 is arranged to monitor the user wearing it and to gather physiological data. When the physiological data gathered meets a predefined trigger condition, the first wearable device 51 initiates a communication protocol pre-established with the second wearable device 52. The first step of this communication protocol involves the first wearable device 51 sending alert signal to the second wearable device 52. Once the second wearable device 52 has acknowledged receipt of the alert signal (by means of a “nod” in accordance with the present techniques as described herein), the first wearable device 51 then begins a data transmission to the second wearable device 52, in which user data generated by the monitor 55 on the basis of the signals received from the physiological sensor 54 and temporarily stored in the data store 56 is transmitted along with associated checksum data, enabling the second wearable device 52 to determine when it has successfully and completely received the data transmission. More detail of the physical mechanism by which the alert signal and the data transmission are sent by given with reference to the figures which follow. It suffices here to identify the transmission electrode 58, worn in contact with the user’s skin, and the floating ground node 59.

The second wearable device 52 comprises a reception electrode 60, worn in contact with the user’s skin, and a floating ground node 61. Reception control circuitry 62 is coupled to the reception electrode 60. Accordingly, the reception control circuitry 62 controls monitoring of the electrical status of the reception electrode to determine when the alert signal is being transmitted by the first wearable device 51. Moreover, when this is the case, the reception control circuitry 62 causes the alert response signal derived from the alert signal received at the reception electrode to be applied to the receiver electrode 60. Once a data transmission from the first wearable device 51 begins, the reception control circuitry 62 passes the data received to the data processing circuitry 63, which is configured to cause the user data transmitted to be stored in the data store 64 and furthermore to determine when the user data has been successfully and completely transmitted by reference to the checksum data which accompanies it. The data processing circuitry 63 of the second wearable device 52 also signals to the user interface 65 as appropriate, for example, in response to the alert signal having been received from the first wearable device 51 (and acknowledged with a “nod”) the user interface 65 can be caused to generate a signal for the user to cause the user to bring the second wearable device 52 into close proximity with the first wearable device 51. When the second wearable device 52 has a display, in the example of it being a smartwatch, then the display may be caused to display a corresponding message to the user. Auditory or vibratory signals may also be generated to gain the user’s attention. However other types of second wearable device are also contemplated here, and the user interface is to be understood accordingly. For example, the second wearable device could be an earpiece worn by the user, in which case the user’s attention is likely to be best gained by means of an auditory or vibratory signal. In other examples, the second wearable device could be a pair of “smart glasses” and the user’s attention could then also be gained by means of a suitable message appearing in their line of sight. Once the data transmission from the first wearable device 51 has been successfully received by the second wearable device 52, the user interface 65 can be further employed to signal to the user that they no longer need to hold the second wearable device 52 in close proximity to the first wearable device 51.

FIG. 4 schematically illustrates a user 70 wearing a first wearable device in the form of a skin adhesive patch 71 and a second wearable device in the form of an ear-mounted device 72 in accordance with some examples. FIG. 5 schematically illustrates a user 75 wearing a first wearable device in the form of the skin adhesive patch 76 and a second wearable device in the form of a pair of glasses 77 in accordance with some examples. As will be understood from the above discussion with reference to FIG. 3, the ear-mounted device 72 may generate an appropriate auditory or vibratory signal in order to signal to the user. Similarly, the pair of glasses 77 may generate an appropriate visual, auditory, or vibratory signal in order to signal to the user. In either case, the user may be directed to move the second wearable device into closer proximity with the skin adhesive patch 71, which may involve the user temporarily taking the ear-mounted device 72 or pair of glasses 77 off, and holding it in close proximity to (or even in contact with) the patch. It can then be replaced thereafter.

FIG. 6 illustrates a simulation of an example distribution of buffer currents at the transmission buffer of a user-worn patch when a further user worn device is responding or not responding in accordance with the present techniques in some examples. The simulation which produced these results was based on the circuit represented in FIG. 9. FIG. 6 shows the results of the transmission buffer current (patch TX buffer current) at the buffer A1 in FIG. 9, representing the transmission electrode in contact with the user’s skin (represented by the “tissue model” in FIG. 9). The simulated system was simulated with typical statistically distributed variations in the physical characteristics of the circuit components represented. The results show the transmission buffer current when the predefined oscillating signal V1 is applied, and the results show clear separation between a first set of simulated resulting transmission buffer currents “with response” and a second set of simulated resulting transmission buffer currents with “no response”. These correspond respectively to whether or not the receiver applies the oscillating response signal V3 (where this is generated from a stored piecewise linear (PWL) function) to the receiver electrode. The effect on the transmission buffer current is clear, in that in the illustrated example the application of the response signal V3 causes the distribution of the transmission buffer currents to move from having a peak at around -11µA to having a peak at around -15µA. Accordingly, it the patch can thus determine an acknowledgement (a “nod”) to the signal it is transmitting, by virtue of a similar shift in the transmission buffer current when the “nod” is received.

FIG. 7 schematically illustrates some components associated with a reception electrode 80 in accordance with some examples. The electrical status of the reception electrode 80 is monitored and receipt of a signal is determined by the reception processing circuitry 81. This identified signal may be an initial alert signal, indicating that a partner device has further data to transmit, or may represent that further data when it is transmitted. In the case that the received signal should be acknowledged, this is achieved by the reception processing circuitry 81 indicating this to the nodding signal injection circuitry 82. The nodding signal injection circuitry 82 is also connected to the reception electrode 80 and is arranged to apply a response signal to the electrode in dependence on the signal received. For example, the response signal applied may match the received signal, especially in terms of its phase and frequency for the purpose of affecting the transmission buffer current measured. Equally an identifiable effect may also be achieved (and hence provide the required “nod”) by matching the received signal in frequency, but applying it in anti-phase in order to create an identifiable effect in the transmission buffer current measured. FIG. 8 schematically illustrates some components associated with a transmission electrode 90 in accordance with some examples. A signal generator 91 is coupled to the electrode 90 and provides the required alert signal in the first instance, when the first wearable device wishes to indicate to the second wearable device that it has data to transmit. A current detector 92 is also coupled to the transmission electrode 90 and measures the current there. In particular, when the alert signal is applied by the signal generator 91 to the transmission electrode 90, the current detector 92 monitors the current and determines when the second wearable device is acknowledging the alert signal by virtue of a shift in the measured transmission buffer current. Similarly, transmission of data from the first wearable device is achieved by means of the signal generator generating an appropriate signal which is applied to the transmission electrode 90 (e.g. by well-known encoding techniques such as amplitude/phase/frequency modulation or amplitude/phase/frequency shift keying). The current detector 92 can also monitor the transmission electrode current during data transmission to identify a shift in the measured transmission buffer current indicative of the second wearable device acknowledging the data transmission.

FIG. 9 schematically illustrates in more detail the configuration of a first wearable device in the form of a skin patch, a tissue model representing the user’s body, and a second wearable receiver device in accordance with some examples. As described above this circuit diagram has been used to simulate the operation of the disclosed system of wearable devices and from which simulation the results shown in FIG. 6 were obtained. The system 100 comprises three main parts, namely a patch (first wearable device) 100, a tissue model (representing the user’s bodily tissue) 102, and a receiver (second wearable device) 103. In the patch 101, the signal generator V1, the resistor R1, and the capacitor C3 enable the patch to provide a signal applied to the transmission buffer A1. Whilst a signal is being applied to the transmission buffer, the set of resistors Rsh, R7 and R8, the capacitors C6 and C7, and the comparator U2 enable the patch to measure the transmission buffer current and to determine (by means of the comparator U2) whether it has shifted by a characteristic amount indicative of a “nod” acknowledgement being generated by the receiver 103. The capacitors C1 and C2 together with the resistors R3, R4, and R5 form the tissue model 102. At the receiver 103, the reception electrode is represented by the capacitor C5 and resistor R2, the electrical status of which is measured by the buffer A2, whose offset is set by the voltage supply V4. The receiver 103 can also inject a response (a “nod”) to the reception electrode via buffer A3, the switching of which is controlled by the PWL file control of oscillating voltage supply V3. The effective ground rails of both patch 101 and receiver 103 are considered to be parasitically connected, represented by the C_isolation capacitor.

FIG. 10 illustrates an example distribution of bit error rates measured in association with body-mediated communication between a first user wearable device and a second user wearable device for a range of distances between the two devices in accordance with some examples. In an experimental configuration to test the bit error rate, a first user wearable device (transmitting the data) and a second user wearable device (receiving the data) are arranged at a range of distances from one another. It can clearly be seen that the bit error rate is in the range of 4.0-5.0% when the wearable devices (specifically their ground planes) are positioned between 10 and 50 cm from one another, but that the bit error rate drops considerably when the wearable devices are even closer, down to approximately 1.0% at 2 cm separation. In the context of wearable devices, where at least one of the wearable devices has a limited power supply and limited ability for data transmission, the advantage of bringing the two wearable devices into close proximity with one another when attempting a data transmission episode can be appreciated. Indeed, as an extreme solution to improving the data transmission fidelity, the user could actually bring the ground plane of each wearable device into contact with one another.

FIG. 11 is a flow diagram showing a sequence of steps which are taken in accordance with the method of some examples. Specifically, the steps shown are example steps taken by a first wearable device which monitors the physiology of the user and takes action to transmit certain data to a second wearable device. The flow can be considered to begin at step 200 where first wearable device is in its normal mode, monitoring at least one physiological quantity with respect to the user. At step 201 it is determined if an anomaly (i.e. a trigger event) with respect to the at least one physiological quantity has been detected. Whilst this is not the case, the flow loops on itself with the first wearable device operating in its normal mode. Once an anomaly is detected, the flow proceeds to step 202, where the first wearable device enters an alert mode in which it seeks to transmit data relating to the anomaly to a second wearable device. This process begins with the next step 203, where the first wearable device transmits a predetermined alert signal to the second wearable device. The first wearable device continues to transmit the predetermined alert signal, until it is determined at step 204 that a first “nod” has been received from the second wearable device. Once the first “nod” is received, the first wearable device enters a critical data transmission mode at step 205, and at step 206 begins transmission of the critical data (i.e. data recorded / generated related to the at least one physiological quantity monitored with respect to the user). The critical data is accompanied by a checksum, enabling the recipient to determine if it has been fully and correctly received. The recipient second wearable device sends a second “nod” to indicate that it has fully correctly received the critical data. Until the second nod is received the flow loops at steps 206 and 207. Once second nod is received then the transmission of the critical data and checksum ends and the flow returns to step 200, for normal physiological monitoring of the user to continue.

FIG. 12 is a flow diagram showing a sequence of steps which are taken in accordance with the method of some examples. Specifically, the steps shown are example steps taken by a second wearable device which can receive certain data from a first wearable device which monitors the physiology of the user. The flow can be considered to begin at step 300 where second wearable device is in its normal mode, which is defined by the usual function of the second wearable device (e.g. acting as a smart watch, acting as an ear-worn device, or acting as a pair of smart glasses). However, the second wearable device does also continually monitor its reception electrode, to identify when an alert signal is received from a first wearable device which monitors the physiology of the user. At step 301 it is determined if an alert signal has been received from the first wearable device and until this is the case the flow loops at step 301. When an alert signal is received the flow proceeds to step 302, where the second wearable device adds the “nod” signal to its reception electrode, in order to affect the transmission electrode current being measured by the first wearable device. Having made this acknowledgement, the flow proceeds to step 303 where the second wearable device waits for a critical data transmission from the first wearable device (e.g. patch). It is then determined, at step 304, whether the checksum accompanying the data transmission is correct (indicating that the data has been fully and correctly received). If the checksum is not correct, then the flow proceeds to step 306 where the second wearable device (e.g. a smart watch in this example) generates a signal to the user to indicate that they should bring the two wearable devices into closer proximity. In this example of a monitoring patch and a smartwatch, the smartwatch indicates to the user that they should bring their wrist wearing the smartwatch closer to their chest (where the patch is worn). After a pause at step 306 it is again determined at step 304 if the checksum is now correct, i.e. that the data transmission has been successful. Once this is the case then the second wearable device transmits a second nod signal at step 307, acknowledging safe reception of the transmitted data. Assuming that the flow has proceeded at least once via the loop of steps 305 and 306, then following step 307 at step 308 the user alert can be ceased and the user can bring their arm back into a normal position. The flow returns to step 300 for normal operation of the second wearable device.

In brief overall summary, wearable devices, systems of wearable devices, and methods of operating the same are disclosed. A first wearable device worn in contact with the user’s skin monitors the user and comprises a transmission electrode in contact with the user’s skin. A second wearable device comprises a reception electrode worn in contact with the user’s skin. The first wearable device can apply an alert signal to the transmission electrode and measures a transmission current at the transmission electrode. The second wearable device monitors an electrical status of the reception electrode and when the alert signal is detected applies an alert response signal to the receiver electrode. The first wearable device identifies application of the alert response signal to the receiver electrode by measurement of a variation of the transmission current at the transmission electrode whilst the alert signal is applied to the transmission electrode.

In the present application, the words “configured to...” are used to mean that an element of an apparatus has a configuration able to carry out the defined operation. In this context, a “configuration” means an arrangement or manner of interconnection of hardware or software. For example, the apparatus may have dedicated hardware which provides the defined operation, or a processor or other processing device may be programmed to perform the function. “Configured to” does not imply that the apparatus element needs to be changed in any way in order to provide the defined operation.

Although illustrative embodiments have been described in detail herein with reference to the accompanying drawings, it is to be understood that the invention is not limited to those precise embodiments, and that various changes, additions and modifications can be effected therein by one skilled in the art without departing from the scope of the invention as defined by the appended claims. For example, various combinations of the features of the dependent claims could be made with the features of the independent claims without departing from the scope of the present invention.

What is claimed is:

1. A system of wearable devices to be worn by a user comprising:
a first wearable device configured to be worn in contact with the user’s skin and to measure at least one physiological quantity with respect to the user, wherein the first wearable device comprises a transmission electrode configured to be worn in contact with the user’s skin; and
a second wearable device configured to be worn by the user, wherein the second wearable device comprises a reception electrode configured to be worn in contact with the user’s skin,
wherein the first wearable device is configured to respond to a trigger event determined by the first wearable device with reference to the at least one physiological quantity by:
applying an alert signal to the transmission electrode; and
measuring a transmission current at the transmission electrode,
wherein the second wearable device is configured to monitor an electrical status of the reception electrode and in response to a determination that the electrical status is indicative of the alert signal being transmitted by the first wearable device to:
apply an alert response signal to the receiver electrode, wherein the alert response signal is derived from the alert signal received at the reception electrode,
and wherein the first wearable device is configured to determine whether the alert response signal has been applied to the receiver electrode by the second wearable device in dependence on a measurement of a variation of the transmission current at the transmission electrode whilst the alert signal is applied to the transmission electrode.

2. The system as claimed in claim 1, wherein the first wearable device is responsive to the determination that the alert response signal has been applied to the receiver electrode by the second wearable device to:
cease applying the alert signal to the transmission electrode; and
initiate transmission of user data derived from measurement of the at least one physiological quantity, wherein the transmission of the user data is encoded in a user data transmission signal applied to the transmission electrode,
wherein the user data transmission signal further encodes checksum data for the user data.

3. The system as claimed in claim 2, wherein the second wearable device is configured to further monitor the electrical status of the reception electrode and to receive the user data transmission signal,
wherein the second wearable device is configured to data process the user data and the checksum data derived from the user data transmission signal and thereby to generate a determination of whether the user data transmission signal has been received correctly.

4. The system as claimed in claim 3, wherein the second wearable device is responsive to the determination indicating that the user data transmission signal has been received correctly to:
apply a user data response signal to the receiver electrode, wherein the user data response is derived from the user data transmission signal received at the reception electrode,
and wherein the first wearable device is configured to determine whether the user data response signal has been applied to the receiver electrode by the second wearable device in dependence on a measurement of a variation of the transmission current at the transmission electrode whilst the user data transmission signal is applied to the transmission electrode.

5. The system as claimed in claim 4, wherein the first wearable device is responsive to the determination that the user data response signal has been applied to the receiver electrode by the second wearable device to:
cease applying the user data response signal to the transmission electrode.

6. The system as claimed in claim 3, wherein the second wearable device is responsive to the determination indicating that the user data transmission signal has not been received correctly to:
generate a user advisory signal to indicate that the user should bring the second wearable device and the first wearable device into closer proximity.

7. The system as claimed in claim 6, wherein the second wearable device is responsive to a subsequent determination indicating that the user data transmission signal has been received correctly after having generated the user advisory signal to:
cease generation of the user advisory signal; and/or
generate a user confirmation signal to indicate that the user data transmission signal has been correctly received so that the user needs no longer hold the second wearable device and the first wearable device in close proximity.

8. The system as claimed in claim 1, wherein the second wearable device is configured to generate the alert response signal to phase match and amplitude match the alert signal received at the reception electrode.

9. The system as claimed in claim 1, wherein the second wearable device is configured to generate the alert response signal to amplitude match and phase shift the alert signal received at the reception electrode.

10. The system as claimed in claim 9, wherein the second wearable device is configured to generate the alert response signal with an opposite phase to the alert signal received at the reception electrode.

11. The system as claimed in claim 1, wherein the first wearable device comprises at least one of:
electronic components arranged on a flexible substrate;
printed electronic components; and
an energy harvesting component to provide a source of electrical power to the first wearable device.

12. A wearable device configured to be worn in contact with a user’s skin comprising:
measurement circuitry configured to measure at least one physiological quantity with respect to the user;
processing circuitry configured to perform data processing on physiological signals received from the measurement circuitry; and
a transmission electrode configured to be worn in contact with the user’s skin,
wherein the processing circuitry is configured to detect to a trigger event in dependence on the physiological signals received from the measurement circuitry and in response to cause:
an alert signal to be applied to the transmission electrode; and
measurement of a transmission current at the transmission electrode,
and wherein the wearable device is configured to determine whether the alert signal has been received by a further wearable device worn by the user in dependence on a measurement of a variation of the transmission current at the transmission electrode whilst the alert signal is applied to the transmission electrode.

13. A wearable device configured to be worn by a user comprising:
a reception electrode configured to be worn in contact with the user’s skin;
voltage monitoring circuitry to monitor an electrical status of the reception electrode; and
voltage application circuitry to apply a response voltage to the reception electrode,
wherein in response to a determination that the electrical status is indicative of an alert signal being transmitted by a further wearable device worn by the user to:
apply an alert response signal to the receiver electrode, wherein the alert response signal is derived from the alert signal received at the reception electrode and is generated to cause a variation in a transmission current for application of the alert signal at a transmission electrode of the further wearable device.

14. A method of operating a system of wearable devices worn by a user comprising:
wearing a first wearable device in contact with the user’s skin, wherein the first wearable device comprises a transmission electrode configured to be worn in contact with the user’s skin;
measuring with the first wearable device at least one physiological quantity with respect to the user;
wearing a second wearable device, wherein the second wearable device comprises a reception electrode configured to be worn in contact with the user’s skin;
responding to a trigger event determined by the first wearable device with reference to the at least one physiological quantity by:
applying an alert signal to the transmission electrode; and
measuring a transmission current at the transmission electrode;
monitoring an electrical status of the reception electrode and in response to a determination that the electrical status is indicative of the alert signal being transmitted by the first wearable device:
applying an alert response signal to the receiver electrode, wherein the alert response signal is derived from the alert signal received at the reception electrode; and
determining whether the alert response signal has been applied to the receiver electrode by the second wearable device in dependence on a measurement of a variation of the transmission current at the transmission electrode whilst the alert signal is applied to the transmission electrode.

15. The method as claimed in claim 14, further comprising:
in response to the determination that the alert response signal has been applied to the receiver electrode:
ceasing applying the alert signal to the transmission electrode; and
initiating transmission from the first wearable device of user data derived from measurement of the at least one physiological quantity, wherein the transmission of the user data is encoded in a user data transmission signal applied to the transmission electrode,
wherein the user data transmission signal further encodes checksum data for the user data.

16. The method as claimed in claim 15, further comprising:
further monitoring the electrical status of the reception electrode and receiving the user data transmission signal;
data processing the user data and the checksum data derived from the user data transmission signal; and
generating a determination of whether the user data transmission signal has been received correctly.

17. The method as claimed in claim 16, further comprising:
in response to the determination indicating that the user data transmission signal has been received correctly:
applying a user data response signal to the receiver electrode, wherein the user data response is derived from the user data transmission signal received at the reception electrode; and
determining at the first wearable device whether the user data response signal has been applied to the receiver electrode by the second wearable device in dependence on a measurement of a variation of the transmission current at the transmission electrode whilst the user data transmission signal is applied to the transmission electrode.

18. The method as claimed in claim 17, further comprising:
in response to the determination at the first wearable device that the user data response signal has been applied to the receiver electrode by the second wearable device, ceasing applying the user data response signal to the transmission electrode.

19. The method as claimed in claim 16, further comprising:
in response to the determination at the second wearable device indicating that the user data transmission signal has not been received correctly, generating at the second wearable device a user advisory signal to indicate that the user should bring the second wearable device and the first wearable device into closer proximity.

20. The method as claimed in claim 19, further comprising:
in response to a subsequent determination at the second wearable device indicating that the user data transmission signal has been received correctly after having generated the user advisory signal:
ceasing generation of the user advisory signal; and/or
generating a user confirmation signal to indicate that the user data transmission signal has been correctly received so that the user need no longer hold the second wearable device and the first wearable device in close proximity.
